(19) United States
(12) Patent Application Publication
(10) Pub. No.: US 2015/0216473 A1
(43) Pub. Date: Aug. 6, 2015

(54) PORTABLE QUANTIFICATION APPARATUS FOR ASSESSING JOINT ACCESSORY MOVEMENT

(71) Applicant: National Yang-Ming University, Taipei (TW)

(72) Inventors: Chung-Huang YU, Taipei (TW); Hsiao-Kuan WU, Taipei (TW)

(21) Appl. No.: 14/615,973

(22) Filed: Feb. 6, 2015

Related U.S. Application Data
(60) Provisional application No. 61/936,488, filed on Feb. 6, 2014.

(51) Int. Cl. A61B 5/00; A61B 19/00; A61H 23/00; A61B 5/11

(52) U.S. Cl. CPC A61B 5/4528; A61B 5/11; A61B 19/46; A61B 5/0051; A61B 5/4824; A61B 5/7475; A61B 5/4836; A61H 23/00; A61B 2019/464; A61B 2562/04; A61B 2562/0252; A61B 2560/0462

(57) ABSTRACT

A portable quantification apparatus for assessing joint accessory movement is disclosed in the present invention. The apparatus includes a reference module, a movement module, a sliding module and a displacement sensor module. The reference module has a first probe and a first force sensor. The movement module has a second probe and a second force sensor. The sliding module is disposed between the reference module and the movement module which allows the movement module to slide alongside with the reference module. When a patient is under a test, the first probe is against one of two adjacent bones of a joint, while the second probe is against the other adjacent bone. The first force sensor and the second force sensor sense a first force and a second force applying to the reference module and the movement module respectively. The displacement sensor module measures the displacement of the movement module over the reference module.

### CROSS-REFERENCE TO RELATED APPLICATIONS

This Non-provisional application claims priority under 35 U.S.C. §119(a) on patent application Ser. No. 61/936,488 filed in Feb. 6, 2014, the entire contents of which are hereby incorporated by reference.

### FIELD OF THE INVENTION

This invention relates to a portable quantification apparatus for assessing joint accessory movement and, more particularly, to a portable quantification apparatus for assessing joint accessory movement by utilizing multiple probes and using one side of a joint as a reference point for further measuring the relative displacement between two bones at two sides of the joint.

### BACKGROUND OF THE INVENTION

Due to insufficient physical activity, pressures from work, many people suffer from joint problems. Thus, their daily lives are affected so as to reduce the quality of life and work efficiency. Therefore, more and more people need to adopt rehabilitation treatments and request the physical therapy of the orthopedic field for recovering their daily lives.

Clinically, the decrease of the functional activities of patients is a common orthopedic problem, and the main cause comes from injured or degenerated contractile tissues, bursa or connective tissues around joints (such as articular capsule, ligaments and so on). The injuries may be resulted from fracture, ligament sprain, muscle spasm, and so on. For example, the frozen shoulder, which is very common, is resulted from the contracture of the connective tissue around the joint. Therefore, the accessory movement of the shoulder joint will be decrease and the range of motion of the patient's shoulder will be limited and so as to seriously affect the daily lives of the patient and further cause compensation to injure other body parts.

The decrease of the mobility of the spine is most often seen joint problems in clinics. The functions of the spine include providing support to the body, buffering external forces applied on the body, protecting internal organs and so on. It is comprised of, from up to down, seven cervical vertebrae, twelve thoracic vertebrae, five lumbar vertebrae, a sacrum and a coccyx to connect with the occipital bone and pelvis. There are two facet joints between adjacent spinal segments which guide and limit movement of the spinal segments. The functions of the facet joints are important but may be disrupted by degeneration, dislocation, fracture or derangement so as to cause pain and the decreased mobility of the spine. That is called “facet joint syndrome”.

The manual therapy is the most effective method for treating the decreased accessory movement of the spine and other joints. The effective manual therapy should be established on an accurate assessment. The assessment of the joint accessory movement is by applying forces, which are parallel to the surface of the joint, to cause the relative movement between adjacent bones for assessing whether the joint and the tissues around the joint are normal or not. By the way, a similar maneuver also can be used for the joint mobilization. That is, different magnitudes, depths and frequencies of force are applied to relieve the patient's pain and increase the mobility of the joint. Such the treatment has a remarkable effect for treating neuromusculoskeletal pain and, more particular, for restoring the mobility of the spine. The assessment and manual therapy for joints has been performed for hundreds of years, however, its effectiveness still depends on the experience of therapists.

So far to assess the accessory movement of a joint in clinic is mainly performed by bare hands. Apparently, it is not reliable and not objective because it is difficult to control the forcing magnitude and the range of the displacement to the same extend for every assessment and different therapists would have different subjective force and displacement ‘feelings/scales’. More importantly, the assessment cannot be objectively recorded for future reference, for evaluating effect of the treatment, and for discussion between therapists.

To solve this problem, there are devices of measuring the applied force and the resulting displacement of the application point, which are able to objectively quantify the ‘stiffness’ of the spine. However, since these devices using one probe, they cannot measurement the joint accessory movement correctly. Because the displacement of other parts of the spine, the depression of the bed, the undulation resulted from the subject's breath, etc. all will contribute to the displacement of the force application point and the one probe approach cannot eliminate these effect.

In addition, some of the devices using motors to apply force on the probe. These devices are thus big and heavy and are not convenient for practical clinical use. More importantly, these motor driven devices may make the patients feel anxious so as to cause undue muscle contraction and effect the accuracy of the assessment. Moreover, for safety concern, the forces applied by motors are limited a small range so that the condition of the joint cannot be fully assessed.

The present invention is to address the need in joint therapy and solves the problems faced by above-mentioned devices.

### SUMMARY OF THE INVENTION

The present invention discloses a portable quantification apparatus for assessing joint accessory movement that measures a displacement between two adjacent bones of a joint caused by the difference of forces applied thereon. The apparatus includes a reference module, a movement module, a sliding module and a displacement sensor module. The reference module has a first probe and a first force sensor. The movement module has a second probe and a second force sensor. The sliding module is disposed between the reference module and the movement module and allows the movement module to slide alongside with the reference module. When a patient is under a test, the first probe is against one of the two adjacent bones and the second probe is against the other one adjacent bone. The first force sensor and the second force sensor sense a first force and a second force applied to the reference module and the movement module respectively. The displacement sensor module measures the displacement of the movement module over the reference module.

In one of the embodiments of the present invention, the sliding module further has a first sliding element and a second sliding element. The first sliding element engages with the reference module in which the first force sensor is disposed between the first sliding element and the first probe. The second sliding element engages with the movement module in which the second force sensor is disposed between the second sliding element and the second probe.

In another one of the embodiments of the present invention, the first sliding element is a slide rail while the second sliding element is a slider. The second sliding element slidably engages with the first sliding element so the movement module is able to move in parallel alongside with the reference module. Preferably, the second sliding element further has another slide rail. The movement module is slidably placed on the slide rail so that it can move toward or away from the reference module. Preferably, the distance between the first probe and the second probe ranges substantially from 18 to 85 mm, but it is not limited to so.

In another one of the embodiments of the present invention, the displacement sensor module has an optical scale and an optical encoder. Preferably, the optical scale is disposed on the reference module and the optical encoder is disposed on the movement module.

In another one of the embodiments of the present invention, the first force sensor and the second force sensor are both load cells.

In another one of the embodiments of the present invention, the first force sensor can be replaced by a switch and the second force sensor is a load cell.

In another one of the embodiments of the present invention, the movement module further has a vibrating motor that serves to adjust power. After the reference module is fixed, the vibrating motor operates to cause the movement module to apply force repeatedly for the joint therapy.

In another one of the embodiments of the present invention, the apparatus disclosed in the present invention further includes a pain index recording module for recording a pain index whenever the patient starts feeling painful or cannot endure the pain.

In another one of the embodiments of the present invention, the apparatus disclosed in the present invention further includes an embedded module which integrates recorded pain index and data from the first force sensor, the second force sensor and the displacement sensor module.

In another one of the embodiments of the present invention, the apparatus disclosed in the present invention further includes a reset button and a capture button. The reset button resets data from the optical encoder. The capture button captures the data from the first force sensor, the second force sensor, the displacement sensor module as well as the embedded module.

The features and advantages of the present invention will be understood and illustrated in the following paragraphs together with FIGS. 1-3.

### BRIEF DESCRIPTION OF THE DRAWINGS

FIG. 1 is a perspective view showing a portable quantification apparatus for assessing joint accessory movement according to a preferred embodiment of the present invention.

FIG. 2 is a front view showing a portable quantification apparatus for assessing joint accessory movement according to a preferred embodiment of the present invention.

FIG. 3 is a flow chart of operating a portable quantification apparatus for assessing joint accessory movement according to a preferred embodiment of the present invention.

### DETAILED DESCRIPTION OF THE INVENTION

The present invention discloses a portable quantification apparatus for assessing joint accessory movement that measures a displacement between two adjacent bones of a joint caused by the difference of forces applied thereon.

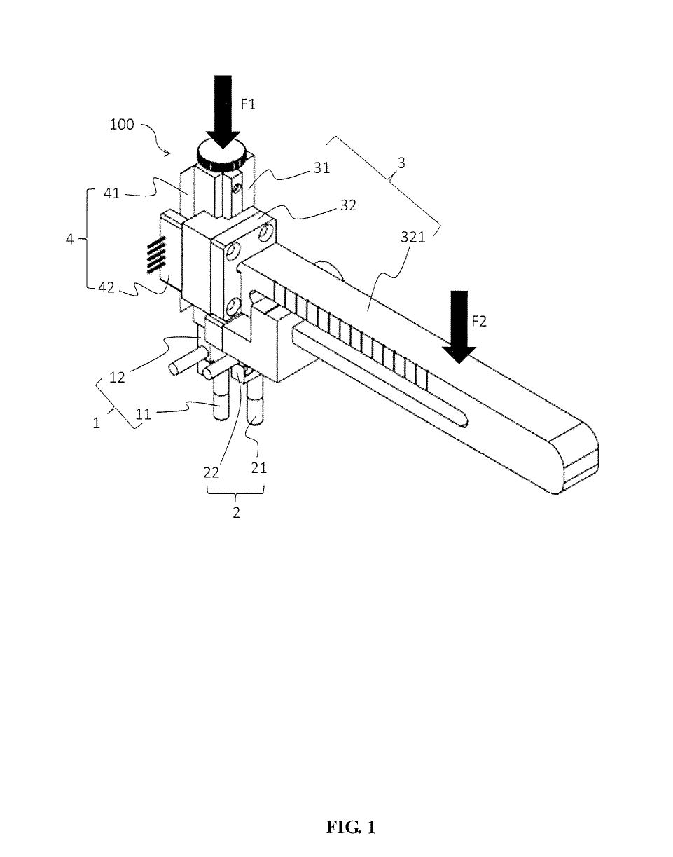

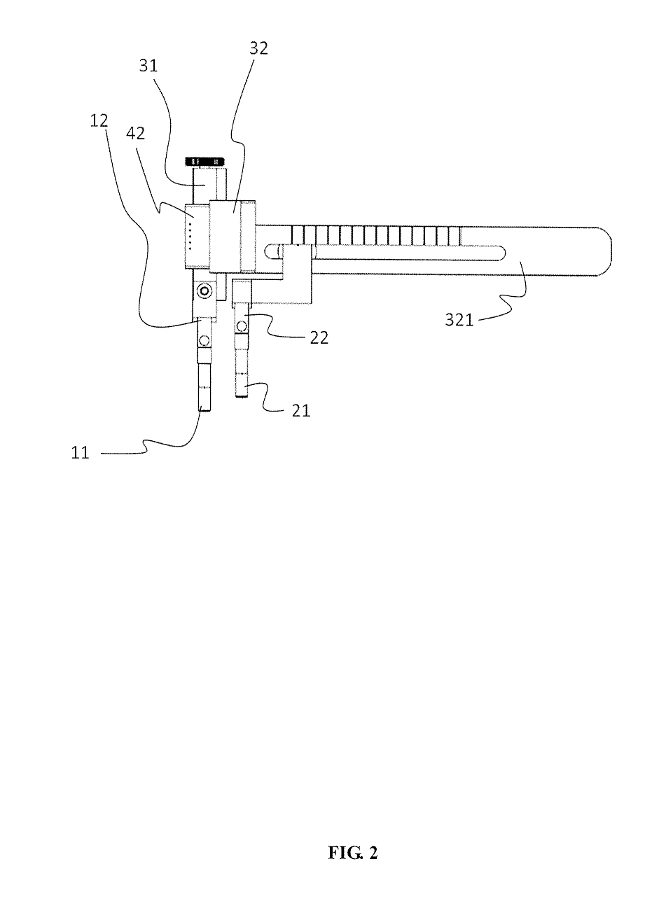

To better understand the concept surrounding the present invention, FIG. 1 is referred to in conjunction with FIG. 2. FIG. 1 is a perspective view showing a portable quantification apparatus for assessing joint accessory movement according to a preferred embodiment of the present invention. FIG. 2 is a front view of the portable quantification apparatus. As can be seen from FIGS. 1 and 2, the apparatus 100 includes a reference module 1, a movement module 2, a sliding module 3 and a displacement sensor module 4. The reference module 1 has a first probe 11 and a first force sensor 12. The movement module 2 has a second probe 21 and a second force sensor 22. In a preferred embodiment, the first force sensor 12 and the second force sensor 22 are both load cells. In another preferred embodiment, the first force sensor 12 is a switch and the second force sensor 22 is a load cell, but they are not limited to so.

The sliding module 3 is disposed between the reference module 1 and the movement module 2 which allows the movement module 2 to be able to slide alongside with the reference module 1. In particular, in the preferred embodiment, the sliding module 3 has a first sliding element 31 and a second sliding element 32. The first sliding element 31 engages with the reference module 1 in which the first force sensor 12 is disposed between the first sliding element 31 and the first probe 11. When a patient is under a test, a first force F1 is applied to the reference module 1 through the first sliding element 31. The first force sensor 12 then measures the first force F1, i.e. the force that applies to one of the adjacent bone contacting with the first probe 11. The second sliding element 32 engages with the movement module 2 in which the second force sensor 22 is disposed between the second sliding element 32 and the second probe 21. The second force sensor 22 measures the second force F2 that applies to the other adjacent bone. Not only the first and the second forces F1 and F2 are measured, the difference between the two is also obtained by the above arrangement of the apparatus 100.

Furthermore, in another preferred embodiment of the present invention, the first sliding element 31 is a slide rail and the second sliding element 32 is a slider. The second sliding element 32 slidably engages with the first sliding element 31. As such, the movement module 2 is able to move in parallel alongside with the reference module 1. It is not intended to be a limitation limit in the present invention, the distance of movement made by the movement module 2 is preferably about 30 mm.

In addition, the second sliding element 32 further includes another slide rail 321. The movement module 2 is slidably placed on the slide rail 321. The movement module 2 is able to move toward or away from the reference module 1 through the slide rail 321 so as to adjust the distance between the first probe 11 and the second probe 21 according to the size of the joint subject to test. Preferably, the distance between the first probe 11 and the second probe 21 ranges from 18 to 85 mm though it is not limited by the present invention. Moreover, the horizontal slide rail 321 may also serve as a handle for the portable quantification apparatus for assessing joint accessory movement disclosed in the present invention. After fixing the distance between the first probe 11 and the second probe 21, a therapist may further apply the second force F2 to the horizontal slide rail 321 to cause the movement module 2 to move in parallel alongside with the reference module 1. The operation will be further described in the following paragraphs.

As can be seen in FIG. 1, the displacement sensor module 4 includes an optical scale 41 and an optical encoder 42. Preferably, the optical scale 41 is disposed on the reference module 1 while the optical encoder 42 is disposed on the movement module 2. The optical scale 41 and the optical encoder 42 serve to measure the displacement of the movement module 2 over the reference module 1. The displacement results from the difference of forces applied to the two adjacent bones of the joint.

It is noteworthy that therapists may perform a joint mobilization by using the apparatus disclosed in the present invention. That is, after fixing the first probe 11, a therapist may force the handle (i.e. the horizontal slide rail 321) repeatedly to cause the second probe 21 to move forward and backward accordingly. By doing so, the joint is therefore eased. As such, the movement module 2 may preferably further include a vibrating motor (whose power is adjustable) to apply forces repeatedly.

In the preferred embodiment, the apparatus of the present invention may further include a pain index recording module, an embedded module, a transmitter, and a user interface (not shown in the figures). Whenever a patient starts feeling painful or cannot endure the pain, he or she may press a recording button. The pain index recording module then records the timing and/or an index of pain for therapists' later reference.

The embedded module integrates data from the first force sensor 12, the second force sensor 22, and the displacement sensor module 4, and the pain index recording module. The data may be further analyzed for later study. Additionally, the user interface preferably equips with a liquid crystal display and/or buttons for therapists' convenience to use and also serves to give notices, such as speeds of applied forces, displacement of joint (i.e. start point and end point), and alerts of pain suffered by the patient. The user interface may also provide real time information about the position of the movement module in the light of the magnitude of the applied force. Such information is helpful for therapists to understand the way in which the force should be applied to the two adjacent bones of the joint. Furthermore, the data can be stored for therapists' later reference.

Preferably, the apparatus disclosed in the present invention may further include a reset button and a capture button (not shown in the figures). When the reset button is pushed, the optical encoder 42 is set as zero displacement. While the capture button is pressed, the data from the first force sensor 11, the second force sensor 21, the displacement sensor module 4, as well as other sensors in the embedded module will be recorded. The reset button and the capture button may be integrated in the user interface; but it is not limited to so.

The operation of the portable quantification apparatus 100 disclosed in the present invention will be discussed in further detail below. Firstly, before a test begins, the first force F1 that applies to the first probe 11 must be adjusted. In more particular, the magnitude of the first force F1 must be able to press the first probe 11 close to the bone in order to reduce the impact resulted from soft tissues. Such magnitude is then fixed so it will not be changed due to the movement module 2. To obtain a proper magnitude of the first force F1, a rigidity test of soft tissue can be performed against the non-joint apophysis of the patient. In detail, making the second probe 21 contact with the skin without applying extra force; subsequently gradually increasing a reference weight that put on the reference module 1 until the displacement does not substantially change. At this moment, the hard portion of the bone is contacted. The minimum threshold value of the reference weight is thereafter adopted as the first force F1.

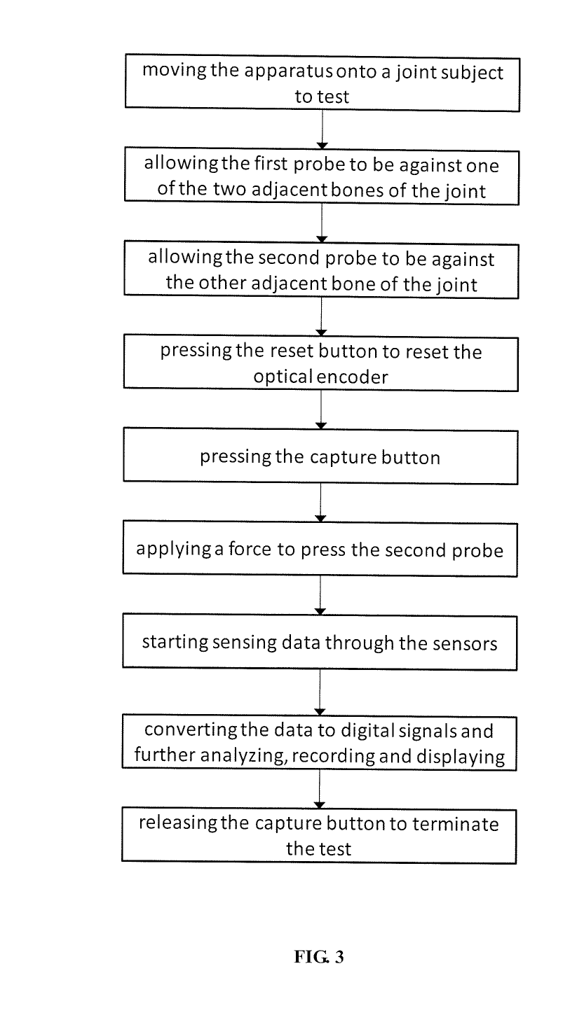

Please refer to FIG. 3, when the apparatus 100 is moved onto a joint subject to test, the first probe 11 is against one of the two adjacent bones of the joint, while the second probe 12 is against the other adjacent bone. Pressing the reset button to reset the optical encoder 42 and then pressing the capture button. The second probe 21 is also pressed. All the sensors in the apparatus 100, such as the first force sensor 12, the second force sensor 22, the optical scale 41, the optical encoder 42, etc. start sensing data. The then obtained data is converted to digital signals through the embedded module for further analysis, recording and display. Finally, releasing the capture button to terminate the test; a result is therefore obtained.

Based upon the above, if the apparatus 100 of the present invention is applied to spine joints, an adjoined vertebra is taken as a reference point for measuring displacement between the adjoined vertebrae. The two probes are placed across a joint. That is, one of the probes serves as a reference end and the other serves as a movement end. The accessory movement of the joint is therefore obtained by the result of the difference of the forces applied to the two ends. As such, therapists are benefited from the present invention. They are able to objectively quantify joint accessory movements by measuring displacements between the two adjacent bones based upon the above mentioned operation. Additionally, pain induced during assessment is also useful as far as clinical concern. The present invention may also serve as tools for the sake of therapist training. To sum up, the advantages of the present invention are as follows: (1) the apparatus is capable of objectively quantifying the forces applied to the two sides of a joint and displacement resulted from it; (2) the distance between the two probes can be adjusted according to the size of the joint subject to test; (3) the confidence level for the manual assessment of the joint accessory movement is increased; (4) the apparatus further assists therapists to better communicate and understand patients' condition; based upon which, therapists are able to pass on the clinical experience; (5) the apparatus provides quantificational notifications during test which may increase the accuracy of the treatment; (6) the apparatus may also analyze how successful the treatment is after the treatment is completed so as to increase the confidence level of the treatment; (7) the apparatus is portable for clinical use; and (8) therapists can learn the joint mobilization technique through the apparatus disclosed herein.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A portable quantification apparatus for assessing joint accessory movement that measures a displacement caused by the difference of forces applied to two adjacent bones of a joint, comprising:
a reference module having a first probe and a first force sensor;
a movement module having a second probe and a second force sensor;
a sliding module is disposed between the reference module and the movement module and slides alongside with the reference module; and
a displacement sensor module;
wherein the first probe is against one of the adjacent bones while the second probe is against the other bone; the first force sensor and the second force sensor sense a first force and a second force applied to the reference module and the movement module respectively, and the displacement sensor module measures the displacement of the movement module over the reference module.

2. The apparatus according to claim 1, wherein the sliding module comprises a first sliding element engaging with the reference module and a second sliding element engaging with the movement module in which the first force sensor is disposed between the first sliding element and the first probe and the second force sensor is disposed between the second sliding element and the second probe.

3. The apparatus according to claim 2, wherein the second sliding element is slidably engages with the first sliding element and the movement module moves in parallel alongside with the reference module.

4. The apparatus according to claim 3, wherein the first sliding element is a slide rail while the second sliding element is a slider.

5. The apparatus according to claim 3, wherein the second sliding element further comprises a slide rail; the movement module is slidably placed on the slide rail and moves toward and away from the reference module.

6. The apparatus according to claim 1, wherein the displacement sensor module comprises an optical scale disposed on the reference module and an optical encoder disposed on the movement module.

7. The apparatus according to claim 6, wherein the apparatus further comprising a reset button for resetting data from the optical encoder.

8. The apparatus according to claim 1, wherein the first force sensor and the second force sensor are load cells.

9. The apparatus according to claim 1, wherein the first force sensor is a switch and the second force sensor is a load cell.

10. The apparatus according to claim 1, wherein the movement module further comprises a vibrating motor whose power is adjustable; the vibrating motor applies force repeatedly to the movement module for the joint therapy.

11. The apparatus according to claim 1, wherein the apparatus further comprising a pain index recording module that records pain index whenever a patient starts feeling painful or cannot endure the pain.

12. The apparatus according to claim 11, wherein the apparatus further comprising an embedded module that integrates the recorded pain index and the data from the first force sensor, the second force sensor and the displacement sensor module.

13. The apparatus according to claim 1, wherein the apparatus further comprising a user interface providing information of the position of the movement module in the light of the magnitudes of the first force and the second force.
